(19) United States
(12) Patent Application Publication
(10) Pub. No.: US 2008/0178435 A1
(43) Pub. Date: Jul. 31, 2008

(54) Personal audio listening device

(75) Inventors: Chun-Hsin Liu, Taipei (TW); Chung-Chun Wu, Taipei (TW); Yu-Chang Hung, Taipei (TW)

Correspondence Address: ROSENBERG, KLEIN & LEE, 3458 ELLICOTT CENTER DRIVE-SUITE 101, ELLICOTT CITY, MD, 21043, US

(73) Assignee: Lite-On Technology Corporation

(21) Appl. No.: 11/715,464

(22) Filed: Mar. 8, 2007

(30) Foreign Application Priority Data
Jan. 29, 2007 (TW) 096201709

(51) Int. Cl. A61F 11/12

(52) U.S. Cl. 24/20 R

(57) ABSTRACT

A personal audio listening device is adapted to be worn on a user's ear. The personal audio listening device includes a housing, a coupling seat, and an ear hook member. The coupling seat is mounted on a surface of the housing, and includes a pivot portion. The coupling seat defines an axis passing through the center of the pivot portion. The ear hook member includes an ear hook portion and a hooking ring. The ear hook portion is substantially C-shaped, and is adapted to be worn on the user's ear. The ear hook portion has a first opening. The hooking ring extends integrally from one end of the ear hook portion to form a C-shape. The hooking ring has a second opening confronting the first opening. The ear hook member engages the pivot portion of the coupling seat through the hooking ring, and is rotatable about the axis.

### CROSS-REFERENCE TO RELATED APPLICATION

This application claims priority of Taiwanese Application No. 096201709, filed on Jan. 29, 2007.

### BACKGROUND OF THE INVENTION

1. Field of the Invention

The invention relates to a personal audio listening device, more particularly to an earphone for use with a mobile communications device.

2. Description of the Related Art

Referring to FIGS. 1 and 2, a conventional ear-hook type earphone 1 includes a housing 11. One side of the housing 11 is provided with a speaker portion 12 adjacent to a top end thereof, and a microphone 13 adjacent to a bottom end thereof. The other side of the housing 11 is provided with a surrounding wall 14 adjacent to the top end, and a wire 15 adjacent to a middle portion thereof and disposed below the surrounding wall 14. The surrounding wall 14 is disposed for fitting of a cap 16 thereon. The surrounding wall 14 is provided with a plurality of spaced-apart teeth 141 protruding from an outer surface thereof. One end of the wire 15 extends into the housing 11, and is connected to the speaker portion 12 and the microphone 13. The other end of the wire 15 is provided with a plug or jack 17 for electrical connection with a mobile phone (not shown). The earphone 1 further includes an ear hook member 18. The ear hook member 18 includes a flexible hooking portion 181 and a gripping portion 182 fitted to one end of the hooking portion 181. The gripping portion 182 engages the surrounding wall 14, with two opposite ends thereof pressed between the cap 16 and the housing 11. The gripping portion 182 is rotatable relative to the surrounding wall 14, and abuts against the teeth 141 on the surrounding wall 14. The gripping portion 181 is disposed for hooking a user's ear, and is formed from a steel wire enveloped by a silicone material so as to be bendable into various shapes to match the user's ear.

However, the method of fabricating the hooking portion 181 of the ear hook member 18, and the need to provide the gripping portion 182 for fitting to one end of the hooking portion 181 so as to engage the surrounding wall 14 render the manufacturing cost high.

### SUMMARY OF THE INVENTION

Therefore, the object of the present invention is to provide a personal audio listening device that is convenient to disassemble, that permits easy angle adjustment, and that can be manufactured at a reduced cost.

Accordingly, the personal audio listening device of the present invention is adapted to be worn on a user's ear. The personal audio listening device comprises a housing, a coupling seat, and an ear hook member. The housing has a surface. The coupling seat is mounted on the surface of the housing, and includes a pivot portion. The coupling seat defines an axis passing through a center of the pivot portion. The ear hook member includes an ear hook portion and a hooking ring. The ear hook portion has a substantially C-shape, and is adapted to be worn on the user's ear. The ear hook portion has a first opening. The hooking ring extends integrally from one end of the ear hook portion, and has a C-shape. The hooking ring has a second opening confronting the first opening of the ear hook portion. The ear hook member clamps the pivot portion via the hooking ring so as to be rotatable about the axis.

Preferably, the hooking ring further has a first end and a second end spaced apart from each other and cooperatively defining the second opening. The first end of the hooking ring is connected to the ear hook portion. The ear hook member further includes an actuated portion extending integrally from the second end of the hooking ring.

Preferably, the hooking ring is in tight fit with the pivot portion of the coupling seat.

Preferably, the ear hook member is formed from a plastic material, and can be alternatively formed from a metal material.

Preferably, the ear hook portion further has a connecting end and a free end spaced apart from each other, and cooperatively defining the first opening. The connecting end of the ear hook portion is connected to the first end of the hooking ring. The actuated portion is substantially C-shaped, and has a third opening oriented in a same direction as the first opening of the ear hook portion.

Preferably, the coupling seat further includes a positioning portion disposed at one side of the pivot portion, a limiting portion disposed at the other side of the pivot portion, and a retaining groove cooperatively defined by the positioning portion, the pivot portion and the limiting portion for retaining the hooking ring.

### BRIEF DESCRIPTION OF THE DRAWINGS

Other features and advantages of the present invention will become apparent in the following detailed description of the preferred embodiment with reference to the accompanying drawings, of which:

FIG. 1 is a perspective view of a conventional ear-hook type earphone;

FIG. 2 is a fragmentary exploded perspective view of the conventional earphone;

FIG. 3 is a perspective view of the preferred embodiment of a personal audio listening device according to the present invention;

FIG. 4 is a perspective view of the preferred embodiment viewed from another angle;

FIG. 5 is an exploded perspective view of the preferred embodiment to illustrate the assembling relationship among a housing, a coupling seat and an ear hook member;

FIG. 6 is a side view of the preferred embodiment;

FIG. 7 is a fragmentary sectional view taken along line 7-7 of FIG. 6, showing how a hooking ring of the ear hook member clamps a pivot portion of the coupling seat; and

FIG. 8 is a side view similar to FIG. 6, showing the ear hook member in another state of use.

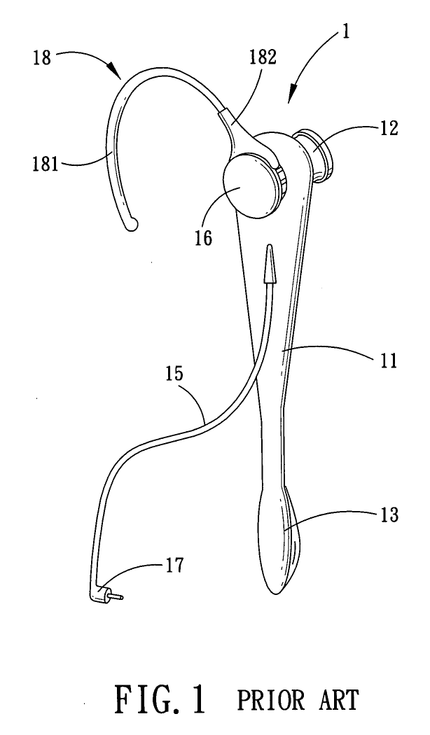

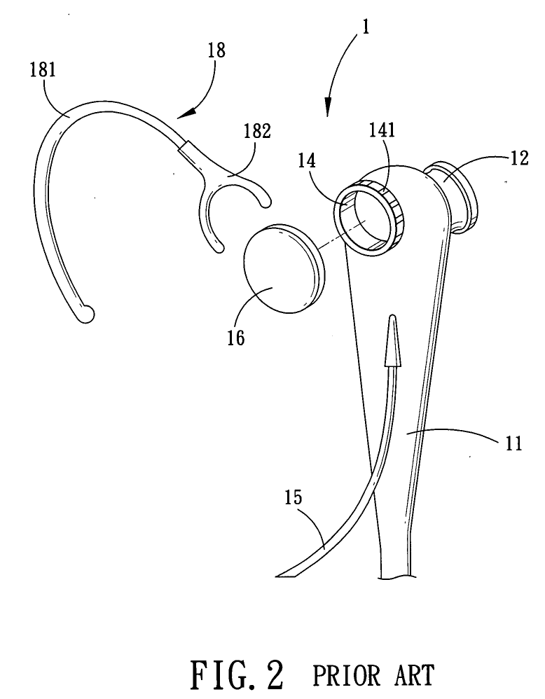

### DETAILED DESCRIPTION OF THE PREFERRED EMBODIMENT

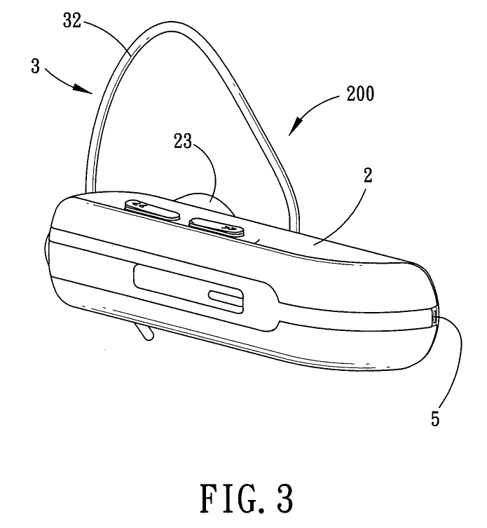

Referring to FIGS. 3, 4 and 5, the preferred embodiment of a personal audio listening device 200 according to the present invention is a wireless earphone having Bluetooth functionality, and includes a housing 2, a coupling seat 4, and an ear hook member 3 adapted to be worn on a user's ear (not shown). It is noted that the personal audio listening device 200 may also be a wired earphone or a hearing aid, and should not be limited to the wireless earphone disclosed herein.

The housing 2 is formed from a plastic material, and includes a surface 21 (i.e., the surface close to the user's cheek), a circular recess portion 22 formed in the surface 21 proximate to a middle portion thereof for receiving the coupling seat 4, and an earplug-like protrusion 23 protruding from the surface 21 at one side of the recess portion 22. The housing 2 has an interior to receive a microphone 5, a speaker (not shown), and a circuit device (not shown) necessary for executing the general functions of a Bluetooth earphone. The microphone 5 is exposed from an end edge of the housing 2, and is provided at the other side of the recess portion 22. The speaker is concealed within the protrusion 23, and the protrusion 23 is adapted to be plugged into the user's ear to permit transmission of sounds from the speaker.

The coupling seat 4 includes a positioning portion 41 in the form of a disk disposed in the recess portion 22, a pivot portion 42 extending axially from an outer side of the positioning portion 41, a limiting portion 43 formed on an outer end of the pivot portion 42, and a retaining groove 44. The positioning portion 41 is retained in the recess portion 22 by glue or adhesive. The positioning portion 41 has an external diameter corresponding to an external diameter of the limiting portion 43 and larger than an external diameter of the pivot portion 42 such that the positioning portion 41, the limiting portion 43, and the pivot portion 42 cooperatively define the retaining groove 44. The coupling seat 4 defines an axis (X) that passes through centers of the positioning portion 41, the pivot portion 42, and the limiting portion 43. Alternatively, the positioning portion 41 of the coupling seat 4 may be connected to the recess portion 22 in the housing 2 by tight fit, or may be bonded to or fixed in the recess portion 22 using any other suitable methods, without being limited to the adhering method disclosed in this embodiment.

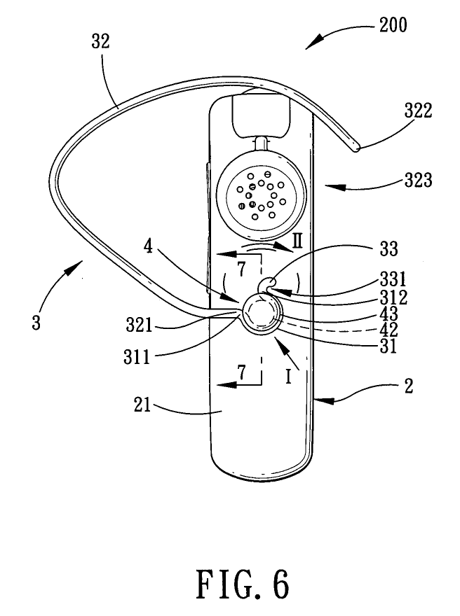

The ear hook member 3 is formed from a resilient material, e.g., formed integrally from a plastic material. The ear hook member 3 includes an ear hook portion 32, a hooking ring 31, and an actuated portion 33. The ear hook portion 32 has a relatively large C-shape, and is adapted to be worn on the user's ear. The ear hook portion 32 has a connecting end 321 and a free end 322 that are spaced apart from each other, and a first opening 323 defined between the connecting end 321 and the free end 322. The hooking ring 31 extends integrally from the connecting end 321 of the ear hook portion 32, and is bent to have a relatively small C-shape. The hooking ring 31 has a first end 311 connected to the connecting end 321 of the ear hook portion 32, a second end 312 connected to the actuated portion 33 and spaced apart from the first end 311, and a second opening 313 defined between the first end 311 and the second end 312 and confronting the first opening 323. A space confined by the hooking ring 31 has a diameter approximating the diameter of the pivot portion 42 of the coupling seat 4. Besides, the second opening 313 has a size smaller than the diameter of the pivot portion 42 of the coupling seat 4. The actuated portion 33 is generally C-shaped, and extends integrally from the second end 312 of the hooking ring 31. The actuated portion 33 has a third opening 331 (see FIG. 6) oriented in the same direction as the first opening 323 of the ear hook portion 32. It is noted that the ear hook member 3 may be formed from a metal material in other embodiments of the present invention, and should not be limited to the plastic material disclosed in this embodiment.

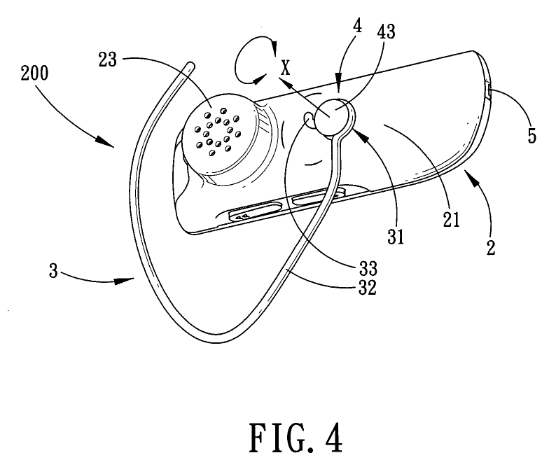

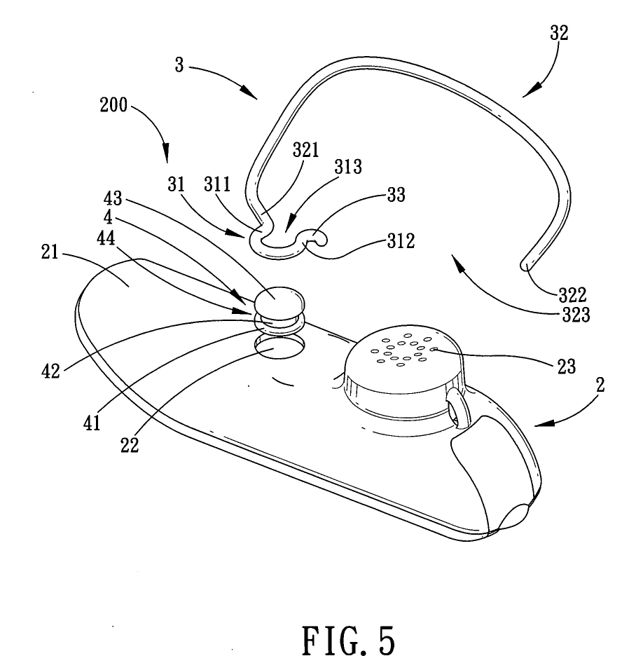

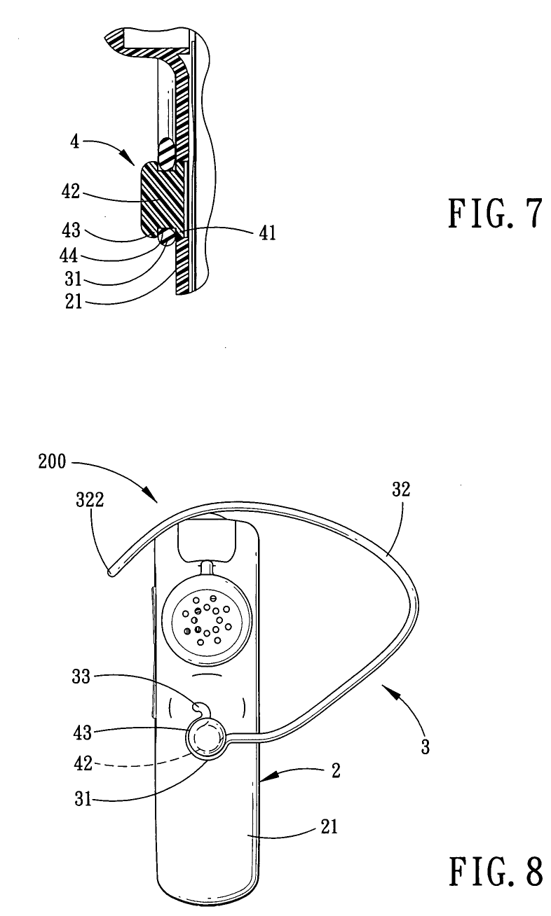

Referring to FIGS. 5, 6, and 7, during assembly, the positioning portion 41 of the coupling seat 4 is adhered fixedly to the recess portion 22 in the housing 2. Next, the ear hook member 3 is assembled onto the coupling seat 4 by first aligning the second opening 313 of the hooking ring 31 with the pivot portion 42 of the coupling seat 4, and then pushing the hooking ring 31 such that the second opening 313 moves in the direction indicated by the arrow (I) in FIG. 6 toward the pivot portion 42. When the first end 311 and the second end 312 of the hooking ring 31 abut against the pivot portion 42, the hooking ring 31 will be slightly deformed, and the second opening 313 will be widened. When an inner lateral side of the hooking ring 31 abuts against an outer surface of the pivot portion 42, due to a resilient restoring action, the hooking ring 31 can clamp the pivot portion 42 firmly to be thereby retained in the retaining groove 44. Thus, the ear hook member 3 is mounted on the coupling seat 4. When the user wants to put on the personal audio listening device 200, the user can rotate the ear hook member 3 relative to the coupling seat 4 about the axis (X) (see FIG. 4) to adjust the angle of the ear hook portion 32 of the ear hook member 3, so that the personal audio listening device 200 can be worn stably on the user's ear.

On the other hand, the user can manipulate the actuated portion 33 to rotate the actuated portion 33 in a clockwise direction indicated by the arrow (II) so that the second opening 313 of the hooking ring 31 is widened, thereby permitting detachment of the hooking ring 31 of the ear hook member 3 from the coupling seat 4. Then, by turning the ear hook member 3 the other way round 180 degrees relative to a direction of extension of the housing 2 in FIG. 6, and by assembling the hooking ring 31 of the ear hook member 3 onto the coupling seat 4, as shown in FIG. 8, the user can wear the personal audio listening device 200 on the other ear.

In sum, the personal audio listening device 200 of the present invention includes the pivot portion 42 of the coupling seat 4 clamped by the hooking ring 31 of the ear hook member 3 which is rotatable about the axis (X). Besides, the arrangement of the ear hook member 3 facilitates assembly and disassembly of the ear hook member 3 to and from the coupling seat 4, as well as angle adjustment of the ear hook member 3. In addition, with the integral formation of the ear hook member 3, the manufacturing cost can also be reduced.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

We claim:

1. A personal audio listening device adapted to be worn on a user's ear, comprising:
a housing having a surface;
a coupling seat mounted on said surface of said housing and including a pivot portion, said coupling seat defining an axis passing through a center of said pivot portion; and
an ear hook member including:
an ear hook portion having a substantially C-shape and adapted to be worn on the user's ear, said ear hook portion having a first opening; and
a hooking ring extending integrally from one end of said ear hook portion and having a substantially C-shape, said hooking ring having a second opening confronting said first opening;
said ear hook member clamping said pivot portion via said hooking ring so as to be rotatable about the axis.

2. The personal audio listening device according to claim 1, wherein said hooking ring further has a first end and a second end spaced apart from each other and cooperatively defining said second opening, said first end of said hooking ring being connected to said ear hook portion, said ear hook member further including an actuated portion extending integrally from said second end of said hooking ring.

3. The personal audio listening device according to claim 1, wherein said hooking ring is in tight fit with said pivot portion of said coupling seat.

4. The personal audio listening device according to claim 1, wherein said ear hook member is formed from a plastic material.

5. The personal audio listening device according to claim 1, wherein said ear hook member is formed from a metal material.

6. The personal audio listening device according to claim 2, wherein said ear hook portion further has a connecting end and a free end spaced apart from each other and cooperatively defining said first opening, said connecting end of said ear hook portion being connected to said first end of said hooking ring, said actuated portion being substantially C-shaped and having a third opening oriented in a same direction as said first opening of said ear hook portion.

7. The personal audio listening device according to claim 1, wherein said coupling seat further includes a positioning portion disposed at one side of said pivot portion, a limiting portion disposed at the other side of said pivot portion, and a retaining groove cooperatively defined by said positioning portion, said pivot portion and said limiting portion for retaining said hooking ring.
